(19) United States
(12) Patent Application Publication
(10) Pub. No.: US 2023/0029047 A1
(43) Pub. Date: Jan. 26, 2023

(54) THERAPEUTIC METHOD FOR TREATING DISABILITIES

(71) Applicant: Delphinia Glbert, Fairlawn, OH (US)

(72) Inventor: Delphenia GILBERT, Fairlawn, OH (US)

(21) Appl. No.: 17/381,609

(22) Filed: Jul. 21, 2021

(51) Int. Cl. G09B 19/00; G09B 5/04; G09B 5/06

(52) U.S. Cl. CPC G09B 19/00; G09B 5/04; G09B 5/065; A61B 5/16

(57) ABSTRACT

A method of treatment for intellectual and developmental disabilities is disclosed herein, wherein the method includes the steps of diagnosing an intellectual or developmental disability in an individual, matching symptoms or characteristics associated with the individual's intellectual or developmental disability, researching what personality traits, interpersonal skills, or life skills are affected by the symptoms or characteristics, providing the individual with a list of topics related to the personality traits, interpersonal skills, or life skills are affected by the symptoms or characteristics, selecting a topic related to the personality traits, interpersonal skills, or life skills are affected by the symptoms or characteristics, assisting the individual in preparing a presentation on the selected topic, preparing the presentation, wherein the presentation is between ten minutes and ninety minutes in length, recording the presentation, and broadcasting the presentation.

### I. BACKGROUND

The present teaching relates to a therapeutic method for treating disabilities, and more particularly to utilizing a media outlet as therapy for treating disabilities.

Intellectual and developmental disabilities (IDDs) are disorders that are usually present at birth and that negatively affect the trajectory of the individual's physical, intellectual, and/or emotional development. Many of these conditions affect multiple body parts or systems. As of 2016, 7.37 million people in the United States had intellectual or developmental disabilities. Intellectual disability starts any time before a child turns 18 and is characterized by problems with both intellectual functioning or intelligence, which includes the ability to learn, reason, problem-solve, and other skills, and adaptive behavior, which includes everyday social and life skills.

The term “developmental disabilities” is a broader category of often lifelong disability that can be intellectual, physical, or both. IDD is the term often used to describe situations in which intellectual disability and other disabilities are present. Examples of developmental disabilities include autism, behavior disorders, brain injury, cerebral palsy, Down syndrome, fetal alcohol syndrome, intellectual disability, and spina bifida.

Developmental disabilities begin anytime during the developmental period and usually last throughout a person's lifetime. Most developmental disabilities begin before a baby is born, but some can happen after birth because of injury, infection, or other factors. Most developmental disabilities are thought to be caused by a complex mix of factors. These factors include genetics; parental health and behaviors (such as smoking and drinking) during pregnancy; complications during birth; infections the mother might have during pregnancy or the baby might have very early in life; and exposure of the mother or child to high levels of environmental toxins, such as lead. For some developmental disabilities, such as fetal alcohol syndrome, which is caused by drinking alcohol during pregnancy, we know the cause. But for most, we do not.

Having a disability does not mean a person is not healthy or that he or she cannot be healthy. Being healthy means the same thing for all of us—getting and staying well so we can lead full, active lives. That includes having the tools and information to make healthy choices and knowing how to prevent illness. Some health conditions, such as asthma, gastrointestinal symptoms, eczema and skin allergies, and migraine headaches, have been found to be more common among children with developmental disabilities. Thus, it is especially important for children with developmental disabilities to see a health care provider regularly.

Often there is no cure, but treatment can help the symptoms. Treatments include physical, speech, and occupational therapy. Special education classes and psychological counseling can also help. Children with Down syndrome, fragile X syndrome, Rett syndrome, and other IDDs can often benefit from therapeutic speech therapy, occupational therapy, and exercises to improve their gross- and fine-motor skills. They might also be helped by special education and attention at school and by the efforts of parents, educators, and health care practitioners to encourage social contact and communication. A variety of treatment options are available to help with the symptoms of autism spectrum disorders. Options may include behavioral, occupational, physical, and speech-language therapy. In addition, educational specialists can help guide the child's school experiences.

Treatment plans for developmental disorders often involve a combination of medical support, skills training, therapy, and other supports. It is important to work closely with your health care professional to figure out the treatment approach that is the right fit. With the right treatment and supports in place, people with developmental disorders can go on to lead very fulfilling lives. Common elements of a treatment plan include medication and medical support, therapy, parent skills training, and support groups. Therapy for the person with a developmental disorder is usually highly structured. It aims to help people to cope better and function at their best. People with developmental disorders are at increased risk of experiencing mental health challenges such as anxiety or depression, which therapy can also help with. Family counseling can provide emotional support and help parents or caregivers learn how to best support their child. Parents of children with developmental disorders may also benefit from individual therapy. These parents tend to experience more stress than other parents. Therapy types to consider include: 1) Cognitive Behavioral Therapy (CBT), which can help change unhelpful thoughts and behaviors. CBT can help people experiencing mental health problems such as anxiety or depression. 2) Family therapy: This helps in many ways, such as providing a forum for family members to understand and learn to support the person with the developmental disorder. You can learn more about Family Systems Therapy here. 3) Creative Arts Therapies: Music, Dance or Art Therapy, for example, can be particularly helpful for children with developmental disorders, for finding new ways of expressing themselves nonverbally. 4) Skills training: Any type of therapy including a skills training component can help people to develop new social skills, coping skills, and skills for participating in work or school, as well as for managing their illness. 5) Applied Behavior Analysis (ABA): ABA is an evidence-based type of therapy commonly used for Autism Spectrum Disorder (ASD). ABA helps build positive behaviors and discourage unhelpful behaviors. It provides skills training to aid communication, social interactions and engaging at school. 6) Mindfulness Practices: Learning mindfulness practices can help to reduce stress levels in parents of children with developmental disorders. 7) Client-centered therapy: Client-Centered Therapy has a couple of adaptations that are helpful for people with developmental disabilities, such as Person-Centered or Child-Centered Play Therapy.

### II. SUMMARY

The present teaching has several benefits over prior treatments.

One aspect of the present teaching is the gathering of individuals with varied disabilities to discuss life as it pertains to them individually.

One aspect of the present teaching includes allowing individuals with varied disabilities to plan and provide a topical broadcast.

One aspect of the present teaching includes allowing individuals with varied disabilities to provide helpful tips for others with disabilities.

One aspect of the present teaching includes allowing individuals with varied disabilities to participate in an informative podcast. Despite their disabilities, the podcast topics can spark a public awareness. It is therapeutic for both the hosts and listeners. Although the hosts are a part of some of the most vulnerable segments of society, the listeners encompass all segments of society.

Still other benefits and advantages of the present subject matter will become apparent to those skilled in the art to which it pertains upon a reading and understanding of the following detailed specification.

### III. DESCRIPTION

The present teaching described herein consists of a therapeutic method of treatment for those with intellectual and developmental disabilities. The treatment regimen begins with identifying a particular individual, or individuals, with either intellectual and/or developmental disabilities. After the particular individual has been identified, that particular individual's disability, or disabilities, are identified. The particular disability, or disabilities, are compared with a list of potential symptoms or characteristics associated with that disability. An analysis is done to determine what types of therapy and treatment best fit the particular list of symptoms or characteristics. The analysis includes researching what personality traits, interpersonal skills, and/or life skills are most affected by the list of symptoms or characteristics.

Once the analysis has been completed, and the personality traits, interpersonal skills, and/or life skills is matched with the list of symptoms or characteristics, a podcast topic is chosen for the individual to deliver. The message is prepared in concert with the disabled individual, and then delivered by the disabled individual. For example, an individual who has ADHD as one of the symptoms of their disability can be paired with the assistance on how to focus. The individual will then prepare and deliver a podcast on tips on how to focus. The preparation and delivery of the message will not only help the individual focus, but also teach public speaking, and can encourage and help the listeners as well. The podcast can be recorded and broadcast. Once the presentation has been broadcast, the individual is evaluated to ascertain any improvement in their symptoms or characteristics. A treatment regimen can be created using a combination of topics, as well as a frequency of presentation. For example, an individual diagnosed with ADHD may be prescribed a treatment regimen of four podcasts on focusing, with the podcasts being created and broadcast once a week. The individual can be evaluated after each presentation, or at the end of the four week treatment regimen. The regimen can be modified with respect to the combination of topics and/or the frequency based upon the evaluation of the individual. The regimen can be adjusted during the regimen, or can be used to create a new, modified regimen for the individual.

As another example, individuals with disabilities frequently suffer from low self-confidence. The particular individual can then be tasked with developing a podcast on how to boost self-confidence. In preparing and delivering an effective message on self-confidence, the individual will help others, but develop their own self-confidence.

It is to be understood that the type of disabilities treated are not limited to any particular disability, and the topics for the messages can be any topic that could assist an individual with a disability. It is also to be understood that the message does not need to be a podcast, but can be any electronically recorded or delivered message format.

The treatments have the aim to challenge active and self-guided activities, which have the highest likelihood to affect an improvement and success for improvement. The treatments can also stimulate comprehension of the families and capabilities of the disabled individuals.

Developmental disabilities are attributable to a cognitive impairment, physical impairment, or both. They manifest during the developmental period from birth to early adulthood, and are likely to continue indefinitely. The life expectancy of most persons with developmental disabilities now approaches that of the general population. According to the neurodiversity model of care, developmental disability is accepted as a valued part of human neurologic diversity. The social model focuses on improving participation in society with accommodations such as adaptive equipment or improvements to the social and physical environment. The goal of health care for patients with developmental disabilities is to improve their well-being, function, and participation in family and community. This treatment method allows direct communication with disabled individual, even those who have communication or cognitive differences. Communication may include speech; sign language; writing; letter boards, voice output devices, pictures, facial expressions, gestures, vocalizations, and behavior. Supported decision making is a paradigm, support practice, and emerging legal structure that focuses on assisting patients with communicating their wants and needs as an alternative to guardianship or power of attorney. When establishing care, a detailed history of baseline traits and characteristics in the areas of cognition and communication should be obtained. These can include neuromuscular function; sensory, function such as vision, hearing, and sensory processing; seizure threshold; mental health; and behavior. With accurate diagnosis, the preparation and delivery of a message can be an effective treatment tool for those with developmental disabilities.

Clause 1—A method of treatment for intellectual and developmental disabilities, the method including the steps of diagnosing an intellectual or developmental disability in an individual, matching symptoms or characteristics associated with the individual's intellectual or developmental disability, researching what personality traits, interpersonal skills, or life skills are affected by the symptoms or characteristics, providing the individual with a list of topics related to the personality traits, interpersonal skills, or life skills are affected by the symptoms or characteristics, selecting a topic related to the personality traits, interpersonal skills, or life skills are affected by the symptoms or characteristics, assisting the individual in preparing a presentation on the selected topic, preparing the presentation, wherein the presentation is between ten minutes and ninety minutes in length, recording the presentation, and broadcasting the presentation.

Clause 2—The method of clause 1, wherein the disability is chosen from the group consisting of autism, behavior disorders; brain injury, cerebral palsy, Down syndrome, fetal alcohol syndrome, learning disability, fragile X syndrome, Rett syndrome, spina bifida, AMID, and Prader-Willi syndrome.

Clause 3—The method of clauses 1 or 2, wherein the personality traits, interpersonal skills, or life skills are chosen from the group consisting of self-confidence, self-esteem, focus, self-control, patience, self-love, kindness, and advocacy.

Clause 4—The method of clauses 1-3, wherein the presentation is chosen from the group consisting of a podcast, online radio program, online video, online live stream broadcast, and digital broadcast.

Clause 5—The method of clauses 1-5, wherein the method further includes the steps of evaluating the individual after recording the presentation and measuring any improvement in the personality traits, interpersonal skills, or life skills.

Clause 6—The method of clauses 1-5, wherein the method further includes the step of creating a treatment regimen, wherein the regimen contains a combination of topics.

Clause 7—The method of clauses 1-6, wherein the regimen contains a frequency of presentations, where in the frequency is chosen from the group consisting of daily, weekly, bi-weekly, monthly, quarterly, and yearly.

Clause 8—The method of clauses 1-7, wherein the method further includes the steps of evaluating the individual after recording each presentation and measuring, after each presentation, any improvement in the personality traits, interpersonal skills, or life skills.

Clause 9—The method of clauses 1-8, wherein the method further includes the step of modifying the regimen based upon the evaluation and measurement.

Clause 10—The method of clauses 1-9, wherein the step of modifying the regimen based upon the evaluation and measurement further includes the step of adjusting the frequency of the presentations.

Clause 11—The method of clauses 1-10, wherein the step of modifying the regimen based upon the evaluation and measurement further includes the step of adjusting the combination of topics.

Clause 12—A method of treatment for intellectual and developmental disabilities, the method including the steps of diagnosing an intellectual or developmental disability in an individual, wherein the disability is chosen from the group consisting of autism, behavior disorders, brain injury; cerebral palsy; Down syndrome, fetal alcohol syndrome; learning disability, fragile X syndrome, Reif syndrome, spina bifida, ADHD, and Prader-Willi syndrome, matching symptoms or characteristics associated with the individual's intellectual or developmental disability, researching what personality traits, interpersonal skills, or life skills are affected by the symptoms or characteristics, wherein the personality traits, interpersonal skills, or life skills are chosen from the group consisting of self-confidence, self-esteem, focus, self-control, patience, self-love, kindness, and advocacy, providing the individual with a list of topics related to the personality traits, interpersonal skills, or life skills are affected by the symptoms or characteristics, selecting a topic related to the personality traits, interpersonal skills, or life skills are affected by the symptoms or characteristics, assisting the individual in preparing a presentation on the selected topic, wherein the presentation is chosen from the group consisting of a podcast, online radio program, online video, online live stream broadcast, and digital broadcast, preparing the presentation, wherein the presentation is between ten minutes and ninety minutes in length, recording the presentation, broadcasting the presentation, creating a treatment regimen, wherein the regimen contains a combination of topics and a frequency of presentations, where in the frequency is chosen from the group consisting of daily, weekly, bi-weekly, monthly, quarterly, and yearly, evaluating the individual after each presentation, and measuring any improvement in the personality traits, interpersonal skills, or life skills.

Clause 13—The method of clause 12, wherein the method further includes the step of modifying the regimen based upon the evaluation and measurement.

Clause 14—The method of clauses 12 or 13, wherein the step of modifying the regimen based upon the evaluation and measurement further includes the step of adjusting the frequency of the presentations.

Clause 15—The method of clauses 12-14, wherein the step of modifying the regimen based upon the evaluation and measurement further includes the step of adjusting the combination of topics.

Non-limiting aspects have been described, hereinabove. It will be apparent to those skilled in the art that the above methods and apparatuses may incorporate changes and modifications without departing from the general scope of the present subject matter. It is intended to include all such modifications and alterations in so far as they come within the scope of the appended claims or the equivalents thereof.

Having thus described the present teachings, it is now claimed:

What is claimed is:

1. A method of treatment for intellectual and developmental disabilities, the method comprising the steps of:
diagnosing an intellectual or developmental disability in an individual;
matching symptoms or characteristics associated with the individual's intellectual or developmental disability;
researching what personality traits, interpersonal skills, or life skills are affected by the symptoms or characteristics;
providing the individual with a list of topics related to the personality traits, interpersonal skills, or life skills are affected by the symptoms or characteristics;
selecting a topic related to the personality traits, interpersonal skills, or life skills are affected by the symptoms or characteristics;
assisting the individual in preparing a presentation on the selected topic;
preparing the presentation, wherein the presentation is between ten minutes and ninety minutes in length;
recording the presentation; and
broadcasting the presentation.

2. The method of claim 1, wherein the disability is chosen from the group consisting of autism, behavior disorders, brain injury, cerebral palsy, Down syndrome, fetal alcohol syndrome, learning disability, fragile X syndrome, Rett syndrome, spina bifida, ADHD, and Prader-Willi syndrome.

3. The method of claim 2, wherein the personality traits, interpersonal skills, or life skills are chosen from the group consisting of self-confidence, self-esteem, focus, self-control, patience, self-love, kindness, and advocacy.

4. The method of claim 3, wherein the presentation is chosen from the group consisting of a podcast, online radio program, online video, online live stream broadcast, and digital broadcast.

5. The method of claim 1, wherein the method further comprises the steps of:
evaluating the individual after recording the presentation; and
measuring any improvement in the personality traits, interpersonal skills, or life skills.

6. The method of claim 1, wherein the method further comprises the step of:
creating a treatment regimen, wherein the regimen contains a combination of topics.

7. The method of claim 6, wherein the regimen contains a frequency of presentations, where in the frequency is chosen from the group consisting of daily, weekly, bi-weekly, monthly, quarterly, and yearly.

8. The method of claim 7, wherein the method further comprises the steps of:
evaluating the individual after recording each presentation; and
measuring, after each presentation, any improvement in the personality traits, interpersonal skills, or life skills.

9. The method of claim 8, wherein the disability is chosen from the group consisting of autism, behavior disorders, brain injury, cerebral palsy, Down syndrome, fetal alcohol syndrome, learning disability, fragile X syndrome, Rett syndrome, spina bifida, ADHD, and Prader-Willi syndrome.

10. The method of claim 9, wherein the personality traits, interpersonal skills, or life skills are chosen from the group consisting of self-confidence, self-esteem, focus, self-control, patience, self-love, kindness, and advocacy.

11. The method of claim 10, wherein the presentation is chosen from the group consisting of a podcast, online radio program, online video, online live stream broadcast, and digital broadcast.

12. The method of claim 8, wherein the method further comprises the step of:
modifying the regimen based upon the evaluation and measurement.

13. The method of claim 12, wherein the step of modifying the regimen based upon the evaluation and measurement further comprises the step of:
adjusting the frequency of the presentations.

14. The method of claim 13, wherein the step of modifying the regimen based upon the evaluation and measurement further comprises the step of:
adjusting the combination of topics.

15. A method of treatment for intellectual and developmental disabilities, the method comprising the steps of:
diagnosing an intellectual or developmental disability in an individual, wherein the disability is chosen from the group consisting of autism, behavior disorders, brain injury, cerebral palsy, Down syndrome, fetal alcohol syndrome, learning disability, fragile X syndrome, Rett syndrome, spina bifida, ADHD, and Prader-Willi syndrome;
matching symptoms or characteristics associated with the individual's intellectual or developmental disability;
researching what personality traits, interpersonal skills, or life skills are affected by the symptoms or characteristics, wherein the personality traits, interpersonal skills, or life skills are chosen from the group consisting of self-confidence, self-esteem, focus, self-control, patience, self-love, kindness, and advocacy;
providing the individual with a list of topics related to the personality traits, interpersonal skills, or life skills are affected by the symptoms or characteristics;
selecting a topic related to the personality traits, interpersonal skills, or life skills are affected by the symptoms or characteristics;
assisting the individual in preparing a presentation on the selected topic, wherein the presentation is chosen from the group consisting of a podcast, online radio program, online video, online live stream broadcast, and digital broadcast;
preparing the presentation, wherein the presentation is between ten minutes and ninety minutes in length;
recording the presentation;
broadcasting the presentation;
creating a treatment regimen, wherein the regimen contains a combination of topics and a frequency of presentations, where in the frequency is chosen from the group consisting of daily, weekly, bi-weekly, monthly, quarterly, and yearly;
evaluating the individual after each presentation; and
measuring any improvement in the personality traits, interpersonal skills, or life skills.

16. The method of claim 15, wherein the method further comprises the step of:
modifying the regimen based upon the evaluation and measurement.

17. The method of claim 16, wherein the step of modifying the regimen based upon the evaluation and measurement further comprises the step of:
adjusting the frequency of the presentations.

18. The method of claim 17, wherein the step of modifying the regimen based upon the evaluation and measurement further comprises the step of:
adjusting the combination of topics.
